(19) United States
(12) Patent Application Publication
(10) Pub. No.: US 2008/0240344 A1
(43) Pub. Date: Oct. 2, 2008

(54) X-ray tomosynthesis device

(76) Inventor: Alfred Reinhold, Wunstorf (DE)

Correspondence Address: SHLESINGER, ARKWRIGHT & GARVEY LLP, 1420 KING STREET, SUITE 600, ALEXANDRIA, VA, 22314, US

(21) Appl. No.: 12/071,810

(22) Filed: Feb. 26, 2008

Related U.S. Application Data
(63) Continuation of application No. PCT/EP2006/010441, filed on Oct. 31, 2006.

(30) Foreign Application Priority Data
Nov. 7, 2005 (DE) 20 2005 017496. 3

(51) Int. Cl. A61B 6/00

(52) U.S. Cl. 378/25

(57) ABSTRACT

X-ray tomosynthesis device includes a target and a device configured for directing a particle beam of electrically charged particles onto the target which emits X-ray radiation for irradiating a sample to be examined when the electrically charged particles strike the target, in use. The target includes at least one support element on which a plurality of mutually spaced target elements are provided, and each mutually spaced target element only partially covers the at least one support element. A deflection device is provided, and the deflection device is configured for causing the particle beam to be deflected in order to strike the plurality of mutually spaced target elements, in use.

### CROSS-REFERENCE TO RELATED APPLICATIONS

This application is a continuation of application no. PCT/EP2006/010441, filed Oct. 31, 2006, which claims the priority of German application no. 20 2005 017 496.3, filed Nov. 7, 2005, and each of which is incorporated herein by reference.

### FIELD OF THE INVENTION

The invention relates to an X-ray tomosynthesis device of the type configured for directing a particle beam of electrically charged particles onto a target which emits X-ray radiation for irradiating a sample to be examined when the electrically charged particles strike the target, in use.

### BACKGROUND OF THE INVENTION

Such devices are generally known, and are used, for example, for examining electronic components, printed circuit modules, or printed circuit boards.

X-ray laminography or tomosynthesis methods are based on the principle of relative motion of an X-ray beam from an X-ray source with respect to an object to be examined. Technical details concerning X-ray laminography or tomosynthesis methods are generally known to one skilled in the art, for example through DE 103 08 529 A1, and therefore are not addressed here in detail.

An X-ray tomosynthesis device is known from DE 103 08 529 A1, comprising an X-ray tube having an X-ray source for generating X-ray radiation for scanning irradiation of an object to be examined, and a holder for the object to be examined. The known device also has an X-ray detector for detecting the X-ray radiation after the object to be examined has been irradiated. In the known device, the object to be examined is held stationary in its holder during the examination, whereas during performance of the laminography or tomosynthesis method the X-ray tube as well as the X-ray detector are moved relative to the object. Similar devices are known from EP 0 683 389 A1, DE 101 42 159 A1, DE 102 42 610 A1, DE 199 51 793 A1, DE 103 17 384 A1, and DE 103 09 887 A1.

A disadvantage of these known devices is that, due to the required motion of the X-ray tube as well as the X-ray detector relative to the object to be examined, significant masses must be moved, which entails significant mechanical complexity and therefore makes the manufacture of the known devices complicated and expensive. This disadvantage is heightened due to the fact that the motion of the masses must be carried out with great precision in order to achieve sufficient image quality, and must also be synchronized with the motion of the X-ray source on the one hand and the motion of the detector on the other hand.

In a departure from known devices, it has previously been proposed to use multiple stationary X-ray detectors instead of one movable X-ray detector. However, such a device still requires motion of the X-ray tube, so that in principle the above-described disadvantages remain.

Furthermore, X-ray laminography and tomosynthesis devices have been proposed in which the X-ray source is stationarily mounted and the object to be examined and the X-ray detector are moved. These known devices as well have the primary disadvantage that significant masses must be moved.

An X-ray tomosynthesis device is known from DE 196 04 802 A1 in which an X-ray source and an X-ray detector are stationarily mounted, whereas a holder for the object to be examined is moved during the examination. Similar devices are also known from DE 197 23 074, U.S. Pat. No. 6,748,046 B2, DE 37 903 88 T1, and DE 102 38 579 A1.

X-ray tomosynthesis devices are also known, for example from DE 103 38 742 A1, in which a stationary X-ray tube having an X-ray source that is movable within the X-ray tube, a stationary holder for the object to be examined, and a stationary X-ray detector are used, and for achieving the required spatial resolution a movable mirror system is used which, after irradiation of the object to be examined, deflects the X-ray radiation onto the X-ray detector corresponding to the particular position of the X-ray beam. A similar device is known from WO 89/04477.

For these devices as well, it is disadvantageous that significant masses must be moved with great precision. The necessary mirror system also entails significant mechanical complexity and makes the manufacture of the known devices costly.

### OBJECTS AND SUMMARY OF THE INVENTION

An object of the present invention is to provide an X-ray tomosynthesis device which is simplified and which can therefore be manufactured more economically, and at the same time allows a high image quality of the X-ray images.

This object is achieved by the inventive X-ray tomosynthesis device according to the invention including a target, and a device configured for directing a particle beam of electrically charged particles onto the target which emits X-ray radiation for irradiating a sample to be examined when the electrically charged particles strike the target, in use. The inventive device further includes that the target has at least one support element on which a plurality of mutually spaced target elements are provided, and each mutually spaced target element only partially covers the at least one support element. A deflection device is provided, the deflection device being configured for causing the particle beam to be deflected in order to strike the plurality of mutually spaced target elements, in use.

The basic concept of the teaching according to the invention is that a motion of the focal spot, at which X-ray radiation is generated when a particle beam of electrically charged particles strikes a target, is achieved without having to move the X-radiation source or the particle source generating the particle beam.

According to the invention, this basic concept is achieved by use of a target having at least one support element, made of a support material, on which a plurality of mutually spaced target elements, each only partially covering the support element, are provided.

According to the invention, the support material and the target material are made of different materials. The target material is selected with respect to an emission of X-ray radiation of a desired wavelength or wavelength range, whereas the support material is selected with respect to its coefficient of thermal conductivity and high transparency to X-ray radiation. According to the invention, the cross section of the support element perpendicular to the direction of radiation is selectively greater than the cross section of the target elements in this direction, so that the target elements cover only a portion of the surface of the support element. The support material also has a lower density, high thermal conductivity, and a specific electrical conductivity, preferably increased by doping, whereas the target material is a high-density material such as tungsten, for example.

Incident electrons are decelerated in the target material over very short distances, thereby preferentially generating shortwave X-ray radiation. In contrast, in the low-density support material incident electrons are decelerated over very long distances, resulting in the generation of longer-wavelength radiation which may be filtered out by use of a suitable filter, for example. As a result, according to the invention the shape, size, and location of the focal spot may be specified by the shape, size, and location of the respective target element.

Since X-ray radiation of a desired wavelength or wavelength range according to the invention is generated exclusively in the respective irradiated target element, and the affected target element thus defines the focal spot of the X-ray tube, the shape and size of the focal spot are no longer dependent on the cross section of the electron beam, but instead depend solely on the cross section of the respective target element, provided that the electron beam consistently irradiates the entire surface of the target during operation of the X-ray tube.

Although X-ray radiation is generated in the support element, this X-ray radiation has a different wavelength or lies in a different wavelength range than the effective radiation generated in the target element, and may thus be easily filtered out.

As a result, according to the invention the focal spot of a device according to the invention may be designed with any given small dimensions, limited only by available microstructuring processes for producing the target elements as micro- or nanostructures.

Since the shape, size, and location of the focal spot are specified exclusively by the shape, size, and location of the respective target element, for a device according to the invention it is possible to omit complex design measures, which are required in conventional devices for stabilizing the shape, size, and location of the electron beam, which in the known devices defines the shape, size, and location of the focal spot. The teaching according to the invention thus allows an X-ray tomosynthesis device to be designed very easily, in which the shape, size, and location of the particular focal spot are highly stable, thus allowing a particularly high image quality when used in imaging methods.

As target material, depending on the particular requirements a material may be used which when bombarded with electrons emits X-ray radiation of a desired wavelength or wavelength range.

When irradiation is performed by particle beam, for example an electron beam, the particular target element acts as an X-ray radiation source. As a result of the target according to the invention having a plurality of mutually spaced target elements, each of which may function as an X-ray radiation source, the device according to the invention has a plurality of potential X-ray radiation sources which are spatially separated from one another and which thus allow the sample to be irradiated at different angles.

To allow one of the target elements to act as an X-ray radiation source, it is only necessary to irradiate this target element with the particle beam so that it emits X-ray radiation. According to the invention, a deflection device is provided for this purpose by means of which the particle beam may be deflected for striking the target elements. Corresponding deflection thus allows the particle beam to be directed at will onto the particular intended target element, which when struck by the electrically charged particles emits X-ray radiation and thus acts as an X-ray radiation source. Thus, by successive irradiation of different target elements with the particle beam it is possible to achieve sequential locations of the X-ray radiation source over time, and thus different irradiation angles during irradiation of the sample.

Since the particle beam may be deflected by suitable coils or coil systems, for example, according to the invention it is possible to change the location of the X-ray radiation source relative to the sample without having to move the X-ray radiation source or parts thereof for this purpose.

Because a change in the location of the X-ray radiation source and thus of the irradiation angle during irradiation of the sample requires no motion of appreciable masses, such a change may be performed without time delays, in a manner of speaking. In this manner, samples may be examined much more quickly using a device according to the invention than with conventional devices. The cycle times for the examination of samples are thus significantly reduced, so that the examination of samples may be designed in a more time-saving and thus a more cost-effective manner.

A further advantage of the device according to the invention is that the device basically functions without mechanical motion of massive components. In this manner the device according to the invention may be constructed with a design that is particularly simple, and thus economical as well as robust.

The shape, size, and number of target elements may be selected within a wide range, depending on the particular requirements. The target elements may be micro- or nanostructures, for example, which are formed on the support element by use of microstructuring processes. Deposition methods, for example, such as three-dimensional additive nanolithography or ion beam sputtering, as well as ablative methods such as electron lithography or etching methods may be used as microstructuring processes. Such methods are generally known to one skilled in the art, and therefore are not addressed here in detail.

According to the invention, the target elements may be provided, for example and in particular, on the surface of the support element. If necessary depending on the particular requirements, however, the target elements may also be embedded in a support element, provided that it is ensured that the electrically charged particles reach the target elements so that the latter emit X-ray radiation.

According to the invention, as an example and in particular a particle source for generating the particle beam of electrically charged particles, a holder for the sample to be examined, and a detector for detecting the X-ray radiation after irradiation of the sample may be stationarily mounted relative to one another. For a correspondingly designed device according to the invention, in the manner described above different irradiation angles may be achieved during irradiation of the sample without having to move one of the above-referenced assemblies. Since the rate of change of the irradiation angle depends solely on the time interval within which the particle beam may be deflected from one target element onto another target element, and such a deflection is possible without time delays, in a manner of speaking, for a device according to the invention the irradiation angle may be changed without time delays, in a manner of speaking. As described above, this allows a particularly high speed in the examination of samples.

According to the invention, a stationary configuration of assemblies relative to one another is understood to mean that these assemblies do not have to be moved relative to one another to achieve different irradiation angles. In this respect it is practical according to the invention to use a target whose surface is at least as large as the maximum surface of the sample to be examined. In this respect it is also practical when the detector used for detecting the X-ray radiation after irradiation of the sample is an X-ray image detector whose input image surface is greater than the maximum surface of the sample to be examined.

If desired depending on the individual requirements, however, according to the invention it is also possible to mount the particle source, the holder for the sample, and the detector so as to be movable relative to one another. However, according to the invention it is not necessary for these assemblies to move relative to one another during the irradiation and examination of a sample.

The X-ray images recorded by the detector may be evaluated, for example, using methods of computer tomography, in particular planar computer tomography, and image processing. Such methods are generally known and are not addressed here in detail.

For a noncircular focal spot, according to the invention the diameter is understood to mean the greatest extension of the focal spot in the focal plane.

Numerical values of coefficients of thermal conductivity refer to room temperature.

It is practical for the deflection device to have at least one coil or coil system and/or at least one electrostatic deflection device. By use of appropriate coils or coil systems or deflection devices it is possible to deflect the particle beam without time delays, in a manner of speaking, for striking the target elements.

One advantageous embodiment according to the invention provides that a particle source for generating the particle beam relative to a detector for detecting the X-ray radiation after irradiation of the sample is stationarily mounted at least during the examination of the sample. Since according to the invention a relative motion between the particle source and the detector is not necessary, this results in a particularly simpler, and thus more economical and robust, design. According to the invention, an examination is understood to mean the recording of multiple X-ray images in direct succession, using different irradiation angles.

In another advantageous embodiment according to the invention, a holder for the sample to be examined is stationarily mounted relative to the particle source and/or the detector, at least during the examination of the sample. In particular in combination with the previously described embodiment, this results in a particularly simpler and thus more economical and robust design in which the particle source, the holder for the sample, and the detector are stationarily mounted relative to one another.

When a large-surface target is used according to the invention, this necessarily requires large deflection angles of the electron beam, and at the same time a large focal length of the electron-optical system is required for the focusing and deflection of the electron beam. This results in distortions of the shape and dimensions of the focal spot, which may cause degradation of the X-ray image quality. To ensure a high image quality and thus allow in particular the examination of increasingly miniaturized samples, for example multilayer printed circuit boards, one extremely advantageous refinement of the teaching according to the invention provides that the target elements have essentially the same contour in a view of the target from above. Since X-ray radiation according to the invention is generated exclusively or almost exclusively by the target elements, and the shape and size of the focal spot in the X-ray tube are thus defined by the shape and size of the respective target elements emitting X-ray radiation, in this embodiment distortions of the shape and dimensions of the cross section of the electron beam remain without affecting the shape and size of the focal spot. Regardless of which of the particular target elements is irradiated by the particle beam, due to the equal contour of the target elements the focal spot always has the same size and dimensions. In this manner the image quality of the X-ray images recorded by the device according to the invention is significantly improved.

The shape and size of the target elements may be selected over a wide range, depending on the particular requirements. In one advantageous refinement of the teaching according to the invention, the target elements are essentially circularly delimited in a view of the target from above. This results in particularly favorable conditions from an optical beam standpoint.

In another advantageous embodiment of the invention, the target is designed as a transmission target.

Another advantageous embodiment of the invention provides that the cross section of the particle beam is selected to be larger than the respective cross section of the target elements, so that the particle beam when directed onto a target element consistently irradiates the entire surface thereof. This ensures that distortions of the shape and dimensions of the cross section of the particle beam remain without affecting the shape and dimensions of the focal spot formed by the particular irradiated target element.

According to another advantageous embodiment of the invention, a control device is provided via which the deflection device may be actuated in such a way that the particle beam irradiates target elements individually or collectively in a predetermined or predeterminable sequence, so that the irradiated target elements emit X-ray radiation according to the predetermined or predeterminable sequence. The actuation of the target elements by the control device may be selected depending on the particular requirements in order to perform, for example, meandering, helical, or linear scanning of the target surface, and thus to achieve a successive activation of the target elements situated on the scanning path with regard to emission of X-ray radiation.

In another advantageous embodiment of the invention, the control device actuates the deflection device in such a way that the particle beam irradiates only one of the target elements. In this embodiment, the particle beam irradiates the target elements individually or collectively in succession so that the target elements successively emit X-ray radiation.

In particular, when the detector used for detecting the X-ray radiation after irradiation of the sample is a large-surface detector composed of multiple small-surface detectors, according to the invention it is also possible to simultaneously irradiate at least two target elements so that at least two X-ray radiation sources are simultaneously active. However, this requires that the X-ray images generated by the various X-ray radiation sources are not superimposed in a manner which degrades the image quality. In this regard, another advantageous embodiment of the invention provides that the control device actuates the deflection device in such a way that at least two target elements are simultaneously irradiated by a particle beam. In one such embodiment, multiple particle sources may be used for generating multiple particle beams.

The support material of which the support element is composed may be selected within a wide range, depending on the particular requirements. The support material has a lower density than the target material, has high thermal conductivity, an increased electrical conductivity, preferably by doping, and high transparency to X-ray radiation.

To achieve a particularly high thermal conductivity, one advantageous embodiment of the invention provides that the support element is composed, at least partially, of a support material having a coefficient of thermal conductivity ≧10 W[/](cm×K), preferably ≧20 W[/](cm×K). This ensures particularly efficient dissipation of the heat resulting from bombardment of the particular target element with high-energy accelerated electrically charged particles, in particular electrons, necessary for the generation of X-ray radiation.

To achieve particularly good heat conduction, in another embodiment of the invention the support material is diamond or contains diamond.

According to another advantageous embodiment of the invention, the support material is doped to increase the electrical conductivity. This refinement is based on the finding that when diamond, for example, is used as support material, although adequate dissipation of the generated heat is ensured, at the same time the target is electrically charged due to the electrical insulation properties of diamond. This refinement is based on the further finding that an electrical charge on the target degrades the image quality due to the fact that an uncontrolled release of electrical charges and reimpingement on the target results in an uncontrolled additional emission of X-ray radiation. When diamond (which is an electrical insulator), for example and in particular is used as the support material, the diamond may be made electrically conductive by doping with a suitable dopant such as a metal or boron, or a metal coating several nanometers thick on the surface facing the particle beam. As a result, electrical charges, for example electrons, may be deflected from the target, thereby reliably avoiding electrical charging of the target which degrades the image quality. Surprisingly, it has been shown that the quality of images recorded in this manner using a device according to the invention may be significantly improved.

In another advantageous embodiment of the invention, the target has a filter which is permeable to the X-ray radiation generated in the target elements, and which at least partially blocks X-ray radiation generated in the target element. This ensures that only X-ray radiation of a desired wavelength or wavelength range irradiates the sample.

Another embodiment of the invention includes an X-ray tomosynthesis device which includes a target, and a device configured for directing a particle beam of electrically charged particles onto the target which emits X-ray radiation for irradiating a sample to be examined when the electrically charged particles strike the target, in use. The inventive X-ray tomosynthesis device also includes a radiation-sensitive detector, the radiation-sensitive detector being configured for recording irradiation images of radiation received at different radiation angles. The irradiation images recorded by the detector are capable of being evaluated by use of computerized tomosynthesis image processing algorithms. The target includes at least one support element on which a plurality of mutually spaced target elements are provided, and each mutually spaced target element only partially covering the at least one support element. Further, a deflection device is provided, and the deflection device is configured for causing the particle beam to be deflected in order to strike the plurality of mutually spaced target elements, in use.

Another embodiment of the inventive X-ray tomosynthesis device includes a particle source, the particle source being configured for generating the particle beam relative to the detector for detecting the X-ray radiation after examination of the sample, and the particle source is stationarily mounted at least during the irradiation of the sample.

The invention is explained in greater detail below with reference to the accompanying highly schematic drawings, in which one exemplary embodiment of a device according to the invention is illustrated. All features that are claimed, described, or illustrated in the drawings, taken alone or in any given combination, constitute the subject matter of the invention, regardless of their summary in the claims or reference to other claims, and regardless of their wording or representation in the description or drawings.

Relative terms, such as up, down, left, and right are for convenience only and are not intended to be limiting.

### BRIEF DESCRIPTION OF THE DRAWINGS

FIG. 1 shows a schematic sectional view of a multilayer printed circuit board;

FIG. 2 shows a schematic sectional view of a multilayer printed circuit board during examination using an X-ray tomosynthesis method according to the PRIOR ART;

FIG. 3 shows, in the same illustration as FIG. 2, a multilayer printed circuit board during examination using another tomosynthesis method according to the PRIOR ART;

FIG. 4 shows a schematic perspective view of one exemplary embodiment of a tomosynthesis device according to the invention;

FIG. 5 shows a schematic side view of the device according to FIG. 4, at a first distance between a sample to be examined and a detector;

FIG. 6 shows the device according to FIG. 4, in the same illustration as FIG. 5, at a second distance between the sample and the detector;

FIG. 7 shows a sectional view of the target for the device according to FIG. 4 in the region of a target element;

FIG. 8 shows a view similar to that of FIG. 7;

FIG. 9 shows a top view of the target according to FIG. 7 in the region of a target element;

FIG. 10 shows a sectional view of an alternative exemplary embodiment of a target for a device according to the invention;

FIG. 11 shows a top view of the target according to FIG. 10;

FIG. 12 shows a top view similar to that of FIG. 11;

FIG. 13 shows an additional top view similar to that of FIG. 11;

FIG. 14 shows a view for illustrating the deformation of the radiation cross section of a particle beam in various spatial positions;

FIG. 15 shows a top view of an alternative exemplary embodiment of a target for the device according to FIG. 4 for irradiation with an electron beam;

FIG. 16 shows a schematic illustration for illustration of a focal spot positioning sequence for the device according to FIG. 4;

FIG. 17 shows a schematic illustration of irradiation images generated by means of the focal spot positioning sequence according to FIG. 16; and

FIG. 18 shows, in the same illustration as FIG. 16, examples of alternative focal spot positioning sequences.

Relative terms, such as left, right, up, and down, are for convenience only, and are not intended to be limiting.

Identical components are provided with the same reference numerals in the drawings. The drawings are highly schematic, and represent strictly schematic diagrams not drawn to scale.

### DETAILED DESCRIPTION OF THE INVENTION

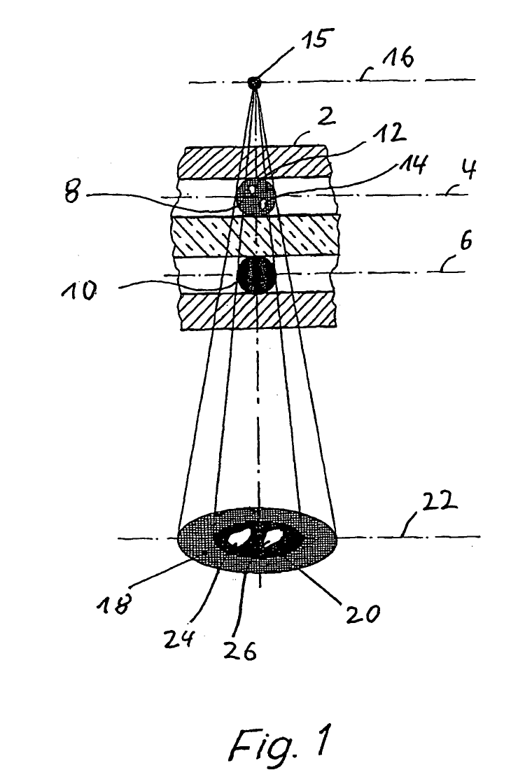

FIG. 1 shows a multilayer printed circuit board 2 during an examination using X-ray radiation in an imaging method. The multilayer printed circuit board 2 has soldering connections, situated in different planes 4, 6, in the form of solder balls, of which two solder balls 8, 10 are shown by way of example in FIG. 1, solder ball 8 having defects in the form of bubbles 12, 14.

The multilayer printed circuit board 2 is examined by irradiation with X-ray radiation which is generated by an X-ray radiation source, the focal spot 15 of which lies in a focal spot plane 16. Irradiation of the solder balls 8, 10 produces irradiation images 18, 20 which are recorded in a detector plane 22 by a detector. This results in irradiation images 24, 26 of the bubbles 10, 12.

As shown in FIG. 1, the irradiation images 18, 20 of the solder balls 8, 10 are superimposed in the detector plane 22. Thus, the irradiation images 24, 26 of the bubbles 10, 12 are discernible on the image recorded by the detector, allowing a determination that one of the irradiated solder balls 8, 10 has defects. However, due to the superimposition of the irradiation images 18, 20 it is not possible to determine in which of the solder balls 8, 10 the bubbles 12, 14 are located. The method illustrated in FIG. 1 is therefore not suitable for assigning and thus localizing defects in the solder balls 8, 10 to the planes 4, 6.

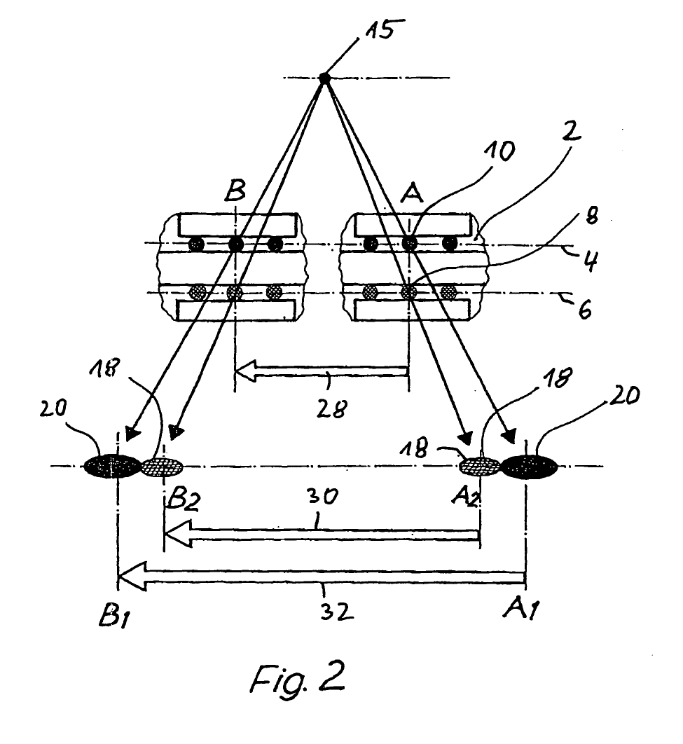

FIG. 2 illustrates a tomosynthesis method according to the PRIOR ART by means of which it is possible to determine not only defects in the solder balls 8, 10, but also to assign the defects to the planes 4, 6 and thus localize them. As shown in FIG. 2, in such a method the multilayer printed circuit board 2 is irradiated obliquely, so that when the irradiation angle is appropriately selected the irradiation images 18, 20 are no longer superimposed in the detector plane 22, but instead are situated at a distance from one another. When the multilayer printed circuit board is in position A, as illustrated in FIG. 2, irradiation images 18, 20 of the solder balls 8, 10 are generated at positions A₂ and A₁, respectively, as shown in FIG. 2.

When the multilayer printed circuit board is moved from position A to position B, parallel to the detector plane 22, as illustrated by an arrow 28 in FIG. 2, the irradiation image 18 for solder ball 8 is then generated at a position B₂, whereas the irradiation image 20 for solder ball 10 is generated at a position B₁.

It can be seen that in the displacement of the multilayer printed circuit board 2 from position A to position B, the irradiation image 18 for solder ball 8 has been displaced by a short distance, indicated by an arrow 30 in FIG. 2, in the detector plane 22, whereas the irradiation image 20 for solder ball 10 has been displaced by a longer distance, indicated by an arrow 32 in FIG. 2, in the detection plane 22. In this manner, when defects in the solder balls 8, 10 are determined an unambiguous assignment may be made as to which of the planes 4, 6 a defective solder ball is located in.

In a corresponding manner, by changing the irradiation angle the other solder balls located in the planes 4, 6 may be examined, and determinations made as to whether these solder balls have a defect. Thus, by use of the method illustrated in FIG. 2 defects may be not only determined but also precisely localized. FIG. 2 illustrates the known method for a three-layer printed circuit board. Of course, while maintaining the basic principle the method may also be used for examining printed circuit boards having more than two layers.

FIG. 3 illustrates an alternative tomosynthesis method according to the PRIOR ART, which differs from the method shown in FIG. 2 by the fact that the multilayer printed circuit board 2 remains stationary during the examination. A change in the irradiation angle, which is necessary to generate the irradiation images 18, 20 at positions A₁ and A₂ on the one hand and positions B₁ and B₂ on the other hand, is achieved in the method illustrated in FIG. 3 by moving the X-ray radiation source, and thus the focal spot 15, in the focal spot plane 16, as represented by an arrow 34 in FIG. 3.

The tomosynthesis methods according to the PRIOR ART illustrated in FIGS. 2 and 3 have the disadvantage that either the multilayer printed circuit board 2 (see FIG. 2) or the X-ray radiation source (see FIG. 3) must be moved during the examination. This motion must be performed with great precision in order to obtain irradiation images of the accuracy required for localization of defects. This is particularly true since the samples to be examined may be highly miniaturized multilayer printed circuit boards. The accuracy necessary for the motion requires a very precise and therefore complex and costly mechanical design for known tomosynthesis devices. A further disadvantage of the known devices is that the examination of samples is time-consuming because each positioning of the X-ray radiation source or sample requires time, and a large number of such positionings must be performed.

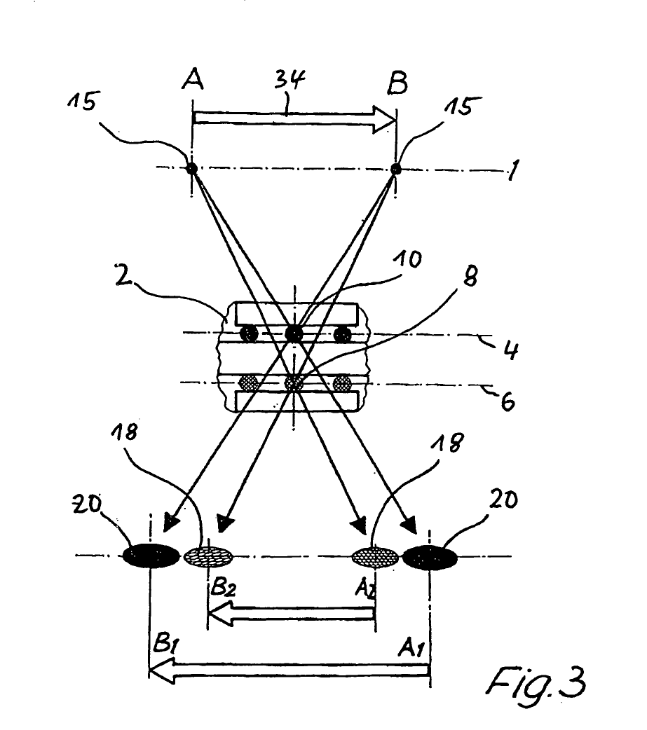

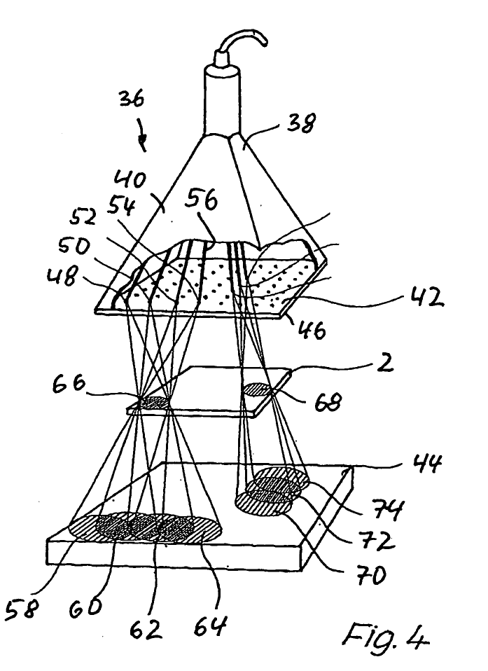

FIG. 4 illustrates an exemplary embodiment of a device 36 according to the invention comprising an X-ray tube 38 having a housing 40 which may be evacuated during operation of the device 36, the interior of the housing accommodating a particle source (not shown in FIG. 4) for generating a particle beam of electrically charged particles. In this exemplary embodiment the particle source is formed by a filament from which electrons are emitted and form an electron beam, which in a known manner, for example by means of a perforated anode, is accelerated in the direction of a target 42. When the high-energy accelerated electrons strike the target 42, X-ray radiation is generated which is used for irradiating the multilayer printed circuit board 2 in a tomosynthesis method. The procedure for generating X-ray radiation is generally known, and therefore is not addressed here in detail.

The device 36 illustrated in FIG. 4 also has a holder for the multilayer printed circuit board 2 as well as a detector 44 that is sensitive to X-rays. In the exemplary embodiment illustrated in FIG. 4, the surface of the target 42 is larger than the surface of the multilayer printed circuit board 2 to be examined. According to the invention it is practical for the surface of the target 42 to be at least as large as the surface of the multilayer printed circuit board 2 to be examined, also referred to below as a sample for short.

In the present exemplary embodiment the input image surface of the detector 44 is larger than the surface of the sample 2 to be examined. The size of the input image surface of the detector 44 is selected such that the generated irradiation images always lie within the boundaries of the input image surface of the detector 44 as a function of the distance of the sample 2 to the target 42, measured in the direction of radiation, and the resulting enlargement of all possible irradiation angles for the sample 2 by means of the X-ray radiation generated by the X-ray tube 38.

According to the invention, the target 42 has a support element 46 on which a plurality of mutually spaced target elements, each only partially covering the support element 46, are provided, which in this exemplary embodiment are arranged in a grid on the support element 46. Of the plurality of target elements, only four target elements having reference numerals 48, 50, 52, 54 are indicated in FIG. 4.

When the electron beam 56 is successively directed onto the target elements 54, 52, 50, 48, these target elements emit X-ray radiation, resulting in irradiation images 58, 60, 62, 64 from a region 66 of the sample 2, in the input image plane of the detector 44, corresponding to the different irradiation angles defined by the respective angle of incidence of the electron beam 56 on the target 42.

For purposes of illustration, FIG. 4 shows that irradiation images 70, 72, 74 result when another region 68 of the sample 2 is irradiated at different irradiation angles.

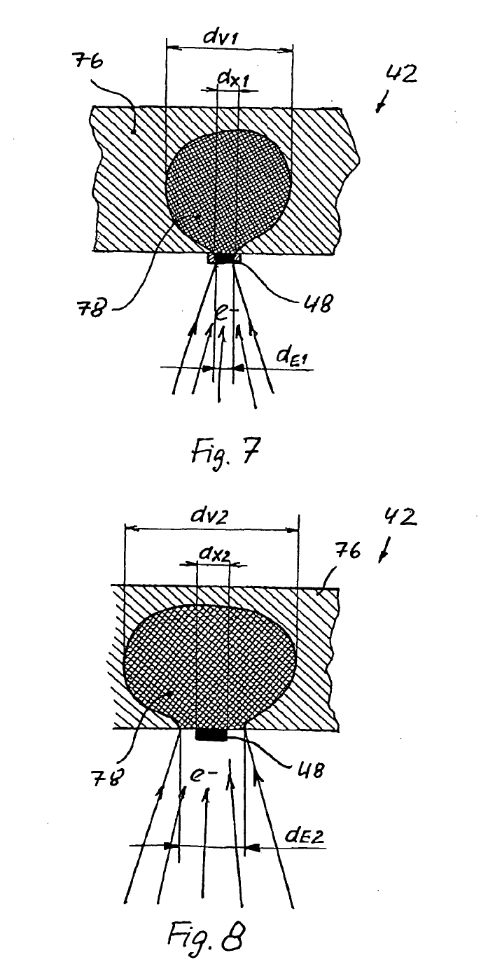

For purposes of illustration, FIG. 4 shows the electron beam 56 in multiple positions at the same time, in which the electron beam irradiates different target elements 48 through 54. In actuality, however, in this exemplary embodiment the electron beam 56 simultaneously irradiates only one of the target elements 48-54 and the additional target elements. When the electron beam 56 strikes one of the target elements, this target element generates X-ray radiation, described in greater detail below with reference to FIGS. 7ff., by means of which the sample 2 is irradiated.

To selectively direct the particle beam onto the individual target elements of the target 42, according to the invention a deflection device is provided via which the electron beam 56 may be deflected for striking the target elements. In the exemplary embodiment illustrated in FIG. 4, the deflection device has a coil system via which the electron beam 56 may be deflected in such a way that it is able to strike each of the target elements of the target 42. If the Z direction, for example, is assumed to the direction of radiation of the electron beam 56, the electron beam 56 may be deflected by the coil system in both the X and Y directions, i.e., in two dimensions. The design and mode of functioning of such coil systems are generally known to one skilled in the art, and therefore are not addressed here in detail.

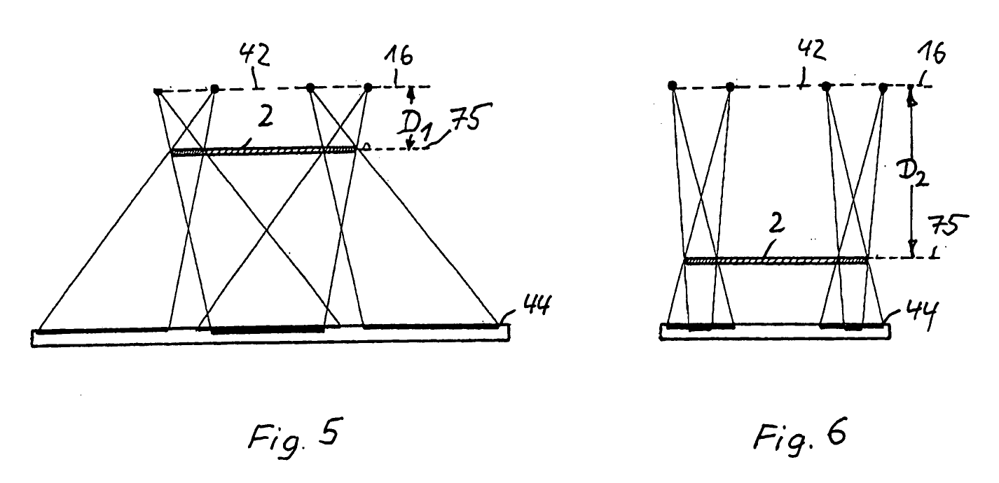

FIGS. 5 and 6 illustrate the dimensioning of the input image surface of the detector 44. FIG. 5 shows the geometric proportions of the beam, at a distance D₁ between the focal spot plane 16 and a plane 75 in which the sample is located. On the other hand, FIG. 6 shows the geometric proportions of the beam resulting from a greater distance D₂ between the focal spot plane 16 and the plane 75. The greater enlargement illustrated for the system according to FIG. 5 compared to the system of FIG. 6 shows that the former requires a larger input image surface of the detector 44.

FIG. 7 shows a schematic sectional view of the target 42 in the region of a target element 48. The target 44 has a support element 76 made of a support material on which the target element 48, made of a target material, and the additional target elements (not shown in FIG. 7) are situated, which emit X-ray radiation when the electron beam 56 is directed onto the particular target element. In principle, the support element 76 is composed of a support material having low density and high thermal conductivity. In the present exemplary embodiment the support material is diamond, which has a coefficient of thermal conductivity ≧20 W/(cm×K).

In this exemplary embodiment the support material is doped to increase the electrical conductivity, and in the present case is provided with boron doping. By making the electrically insulating support material itself electrically conductive by doping, electrical charges are able to flow from the support element 76, thereby avoiding electrical charging of the support element 46 and thus of the target 42.

The target element 48 is composed of a high-density material, tungsten in the present exemplary embodiment, which emits X-ray radiation when bombarded with electrically charged particles, in particular electrons. The following discussion refers only to the target element 48; the other target elements have a corresponding design.

Not shown in FIG. 7, and therefore discussed here, is the fact that the target element 48 is essentially circularly delimited as viewed from the top. The diameter of the target element 48, which, as explained in greater detail below, defines the diameter of the focal spot of the X-ray tube 38, is selected according to the desired resolution of the images obtained by irradiation of the sample 2 and recorded by the detector 44. The target element 48 may, for example, be a micro- or nanostructure which is formed on the support element 4 by means of a microstructuring process and which has a diameter that is dependent solely on the accuracy of the microstructuring process used, and which may be equal to or less than approximately 1,000 nm.

As the result of irradiation of the target element 48 with electrons by directing the electron beam 56 onto the target element 48, the electrons in the target element 48 are decelerated over very short distances, thereby generating shortwave X-ray radiation. In contrast, for the lower-density support material of the support element 76 incident electrons are decelerated over very long distances, resulting in the generation of longer-wavelength radiation. FIG. 7 illustrates a case in which the electron beam having a diameter d_{E1} strikes the target element 48, diameter d_{E1} in this case being smaller than the diameter of the target element 48. The deceleration of the electrons in the target element 48 results in shortwave X-ray radiation having a source diameter d_{X1} which is equal to or less than the diameter of the target element 48. The electrons penetrating through the target element 48 into the lower-density support material of the support element 76 are decelerated over very long distances within the deceleration volume 78 inside the support element 76, resulting in predominantly longwave radiation which can be retained by suitable filters, so that the only shorter-wavelength portion of the radiation which is effective is that originating from the support element 48, which according to the invention only partially covers the surface of the support element 76.

FIG. 8 illustrates a case in which the diameter d_{E2} of the cross section of the electron beam is much greater than the diameter of the target element 48. In this case as well, the predominantly shortwave radiation is generated in the defined delimited target element 48 having the diameter d_{X2}, whereas the electrons within the deceleration volume 78 which penetrate the lower-density support material of the support element 76 result in longer-wavelength radiation, which may be filtered so that only the shorter-wavelength λ-ray radiation, originating from the target element 48, of a defined wavelength or wavelength range is effective for irradiating the sample 2.

A comparison of FIGS. 7 and 8 shows that the shape, size, and location of the focal spot of the X-ray tube 38 depend exclusively on the shape, size, and location of the target element 48 or one of the other target elements onto which the electron beam 56 is directed, and not on the shape, size, and location of the cross section of the electron beam.

FIG. 9 shows a view of the target according to FIG. 8, showing that the diameter d_{E} and thus the cross section 80 of the electron beam 56 is larger than the diameter d_{M} and thus the cross section of the target element 48. As explained with reference to FIGS. 7 and 8, however, only the cross section of the target element 48 perpendicular to the surface of the target 42 is the determining factor for the cross section of the focal spot of the X-ray tube 38.

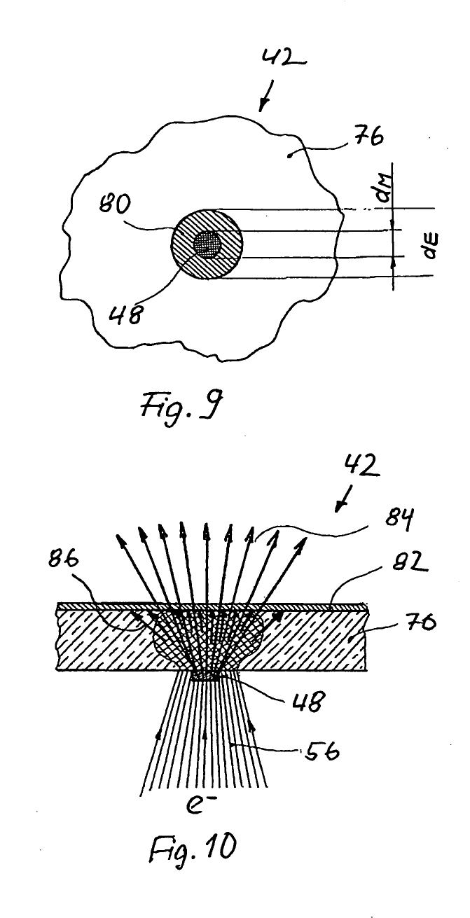

FIG. 10 illustrates an alternative embodiment of a target 42 for the device 36 according to the invention designed as a transmission target, which differs from the exemplary embodiments according to FIGS. 7 and 8 in that the support element 76 has a radiation filter 82, on its side facing away from the target element 48, which is permeable to X-ray radiation 84 generated in the target element 48 but which substantially absorbs X-ray radiation 86 generated in the support element 76. The radiation filter 82 may be provided by an aluminum foil, for example.

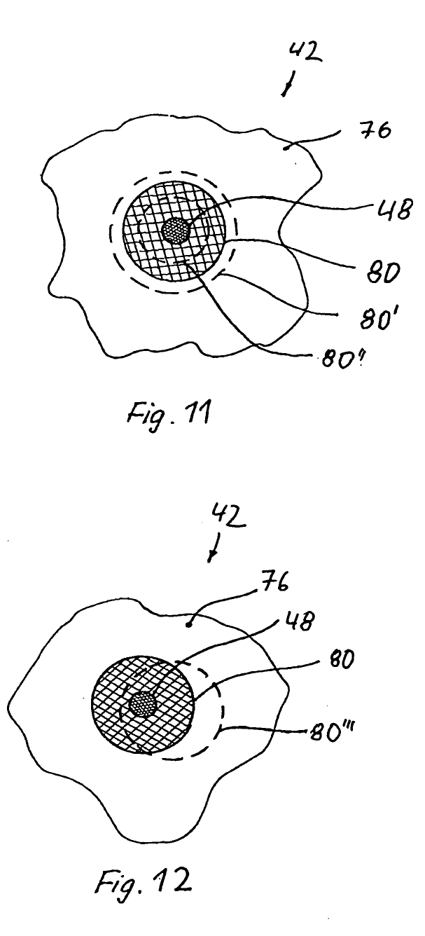

FIG. 11 shows a preset cross section of the electron beam 56, designated by reference numeral 80, whereas a cross section of the electron beam 56 that is enlarged due to interfering effects is designated by reference numeral 80′ and a cross section of the electron beam 56 that is reduced due to interfering effects is designated by reference numeral 80″. Since the cross section of the focal spot of the X-ray tube 38 depends exclusively on the cross section of the target element 48, which is constant, corresponding fluctuations of the cross section of the electron beam 56 have no influence on the cross section of the focal spot, provided that the target element 48 is irradiated over its entire surface by the electron beam 56.

As shown in FIG. 12, the same applies to a lateral displacement of the electron beam 56 transverse to its radiation axis to a position 80″′, since in this position of the electron beam 56 as well, the target element 48 is still impinged over its entire surface by the electron beam 56.

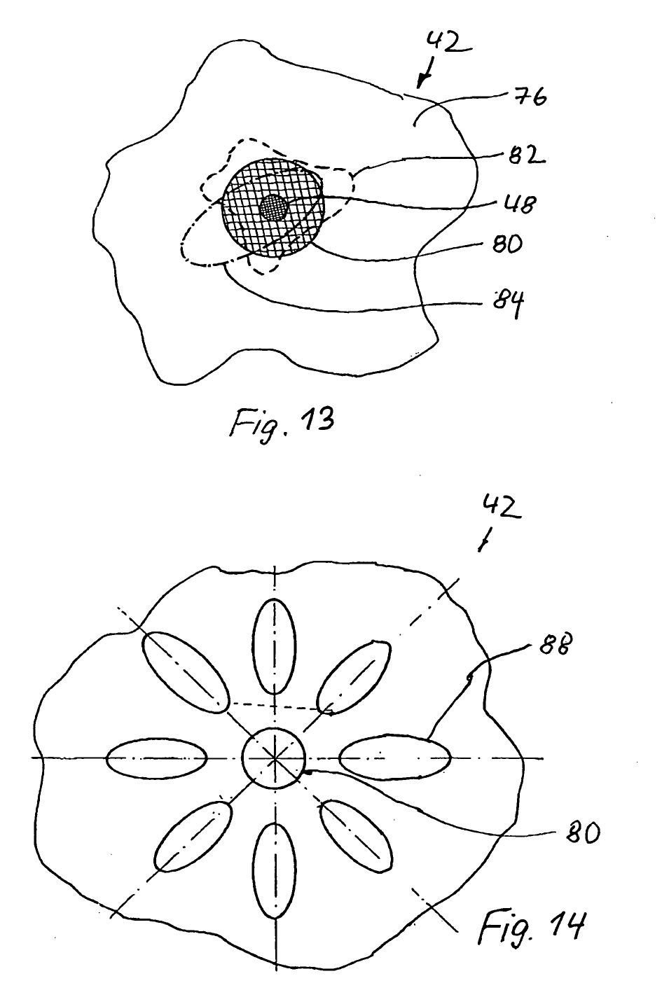

FIG. 13 shows that changes in the cross section of the electron beam 56 also have no effect on the cross section of the focal spot, provided that after a change in cross section of the electron beam 56 the target element 48 is still irradiated over its entire surface. Shown in FIG. 13 by way of example only are two distorted cross sections of the electron beam, designated by reference numerals 82 and 84. Since the cross section of the focal spot of the X-ray tube 38 depends solely on the cross section of the target element 48, which is constant and stable in position, changes in the cross section of the electron beam 56 do not result in degradation of the X-ray image quality for the device 36 according to the invention.

An inspection of FIGS. 11 through 13 shows that cross-sectional changes and displacements of the electron beam 56 do not have any effect on the cross section and location of the focal spot. Accordingly, complicated design measures, by means of which the shape, size, and point of incidence of the electron beam on the target must be stabilized in conventional X-ray tubes used in imaging methods in order to achieve adequate image quality, may be omitted in the X-ray tube 48.

FIG. 14 illustrates exaggerated distortions of the cross section of the electron beam 56 which occur at various angles of incidence of the electron beam 56 on the target 42. Reference numeral 80 denotes the effective cross section of the electron beam 56 on the target 42 when the electron beam strikes the target 42 at an angle of less than 90°. On the other hand, when the electron beam 56 strikes the target 42 at an angle that is different from 90°, the effective cross section is ellipsoidally distorted. FIG. 14 shows ellipsoidally distorted cross sections in various deflection positions of the electron beam 56 corresponding to different irradiation angles; for the sake of clarity only one of the distorted cross sections is provided with reference numeral 88. For a conventional X-ray tube, correspondingly distorted cross sections 88 of the electron beam would result in a distorted geometry of the focal spot of the X-ray tube, thus significantly degrading the image quality.

FIG. 15 shows the conditions resulting in a target 42 according to the invention, whereby in addition to the target element 48 target elements 92, 94, 96, 98, 100, 102, and 104 are shown by way of example only. It is shown that the target elements 48 and 92 through 104, each viewed in the top view of the target 42, have a circularly delimited contour in the exemplary embodiment illustrated. FIG. 15 further shows that the cross section of the target elements 48 and 92 through 104 in each case is smaller than the distorted or undistorted cross section of the electron beam 56, so that in the respective deflection position of the electron beam 56 the particular target element 48 or 92 through 104 is irradiated over its entire surface. Since the shape, size, and location of the particular focal spot, as explained above, depend exclusively on the shape, size, and location of the respective irradiated target element 48 or 92 through 104, an ideal circular focal spot results in each case. In this manner, by use of the device 36 according to the invention great precision is obtained with regard to the shape, size, and location of the focal spot, even for large angles of deflection which are desirable in principle, thus resulting in a particularly high image quality.

The mode of operation of the device 36 according to the invention is as follows:

For examination of the sample 2, the sample is held by means of the holder at a distance from the target 42 for the X-ray tube corresponding to the desired enlargement. During the examination of the sample 2 the X-ray tube 38, sample 2, and detector 44 are mounted so as to be stationary relative to one another.

If region 66, for example, of the sample 2 is to be examined, the electron beam 56 is first directed onto the target element 54 via the deflection device, so that when the electrons strike the target element 54 in the above-described manner X-ray radiation is emitted, by means of which the region 66 is irradiated, so that the irradiation image 58 is generated and recorded by the detector 44. To achieve the various irradiation angles necessary in a tomosynthesis method (see FIG. 3), the electron beam 56 is deflected via the deflection device in such a way that the electron beam subsequently strikes the target elements 52, 50, 48, for example, thereby generating the irradiation images 60, 62, 64.

Since according to the invention the location of the X-ray radiation source relative to the sample 2 is changed solely by directing the electron beam 56 onto different target elements, according to the invention the different irradiation angles required may be achieved without any mechanical motion of the X-ray radiation source, sample 2, and detector 44. Since the deflection of the electron beam 56 by means of the coil system, and thus the transition from one target element to another target element, may occur without time delays, in a manner of speaking, the sample 2 may be examined at extremely high speeds not attainable with known tomosynthesis devices. Furthermore, since the different irradiation angles required may be achieved without massive components, the device 36 according to the invention also is particularly simple in design and thus particularly economical and robust.

The irradiation images recorded by the detector 44 are evaluated in a manner generally known to one skilled in the art by use of computerized tomosynthesis algorithms and image processing, and therefore are not addressed here in detail.

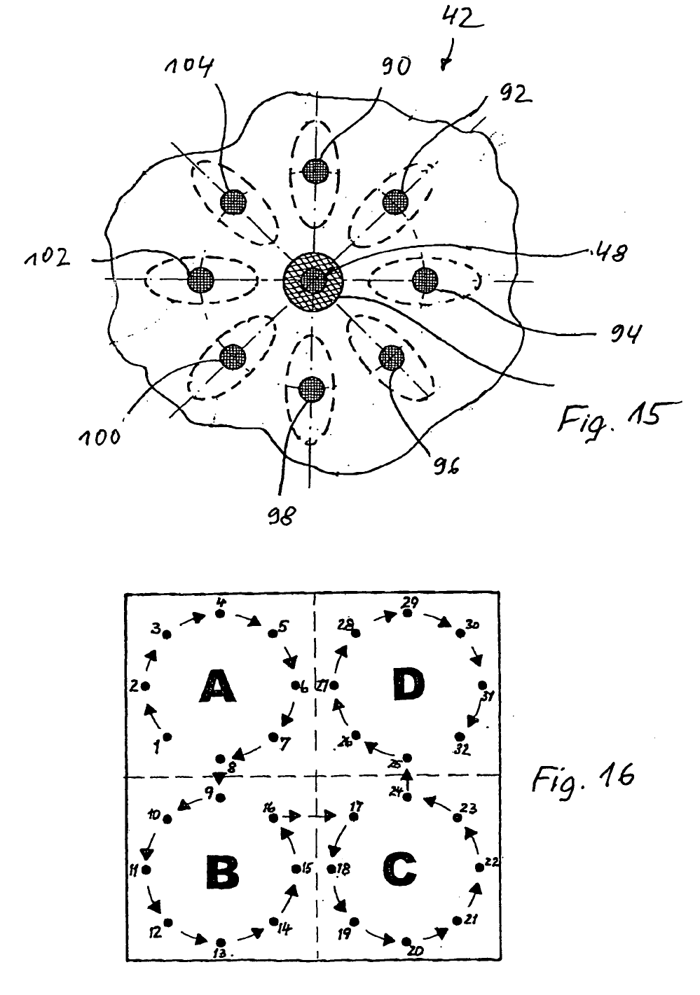

Thus, according to the invention the focal spot of the X-ray tube 38 is positioned by directing the electron beam onto one of the target elements. FIG. 16 illustrates corresponding focal spot positioning sequences for a sample 2 divided into four sections A, B, C, and D. It is seen from FIG. 16 that in the exemplary embodiment illustrated the focal spot in each case is positioned along a circular path.

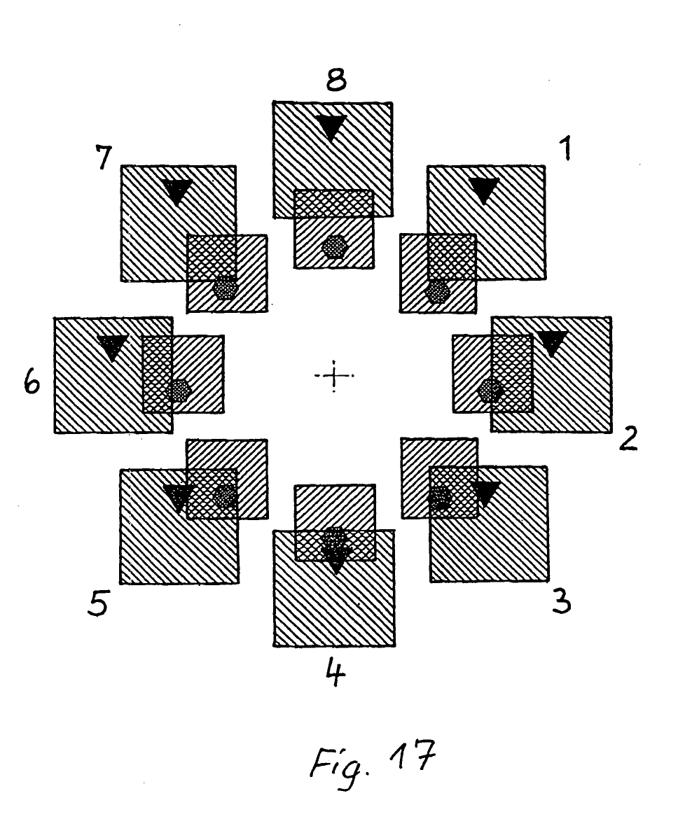

FIG. 17 illustrates an X-ray image sequence resulting from a focal spot positioning sequence according to FIG. 16, with reference to section A of sample 2. In FIG. 17 an upside-down black triangle represents a detail in a plane of the sample near the focal spot, whereas a gray hexagon represents a detail in a plane of the sample 2 far from the focal spot. An evaluation of the irradiation images thus recorded allows defects in the sample 2 to be localized in the manner described above with reference to FIG. 2, using tomosynthesis and image processing algorithms.

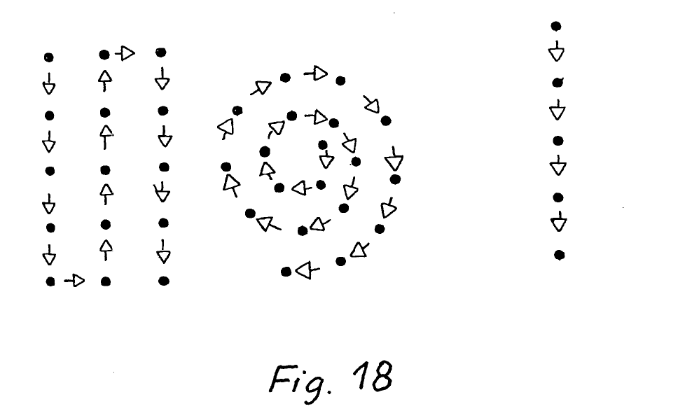

FIG. 18 represents alternative focal spot positioning sequences strictly by way of example. The left portion of FIG. 18 shows meandering positioning, the middle portion shows helical positioning, and the right portion shows linear positioning of the focal spot.

While this invention has been described as having a preferred design, it is understood that it is capable of further modifications, and uses and/or adaptations of the invention and following in general the principle of the invention and including such departures from the present disclosure as come within the known or customary practice in the art to which the invention pertains, and as may be applied to the central features hereinbefore set forth, and fall within the scope of the invention or limits of the claims appended hereto.

1. X-ray tomosynthesis device, comprising:
a) a target;
b) a device configured for directing a particle beam of electrically charged particles onto the target which emits X-ray radiation for irradiating a sample to be examined when the electrically charged particles strike the target, in use;
c) the target including at least one support element on which a plurality of mutually spaced target elements are provided, and each mutually spaced target element only partially covering the at least one support element; and
d) a deflection device provided, the deflection device being configured for causing the particle beam to be deflected in order to strike the plurality of mutually spaced target elements, in use.

2. Device according to claim 1, wherein:
a) the deflection device includes at least one of a coil, a coil system, and at least one electrostatic deflection device.

3. Device according to claim 1, wherein:
a) a particle source configured for generating the particle beam relative to a detector for detecting the X-ray radiation after examination of the sample is stationarily mounted at least during the irradiation of the sample.

4. Device according to claim 3, wherein:
a) a holder for the sample to be examined is stationarily mounted relative to one of the particle source and the detector, at least during the examination of the sample.

5. Device according to claim 1, wherein:
a) the plurality of mutually spaced target elements have substantially the same contour in a view of the target from above.

6. Device according to claim 1, wherein:
a) the plurality of mutually spaced target elements are substantially circular in a view of the target from above.

7. Device according to claim 1, wherein:
a) the target is configured as a transmission target.

8. Device according to claim 1, wherein:
a) the cross section of the particle beam is configured to be larger than the respective cross section of the target elements, so that the particle beam when directed onto a target element, in use, consistently irradiates the entire surface thereof.

9. Device according to claim 1, wherein:
a) a control device is provided by which the deflection device may be actuated in such a way that the particle beam irradiates the target elements one of individually and collectively in one of a predetermined and a predeterminable sequence, so that the irradiated target elements emit X-ray radiation according to a respective predetermined and predeterminable sequence.

10. Device according to claim 9, wherein:
a) the control device actuates the deflection device in such a way that the particle beam irradiates only one of the target elements.

11. Device according to claim 9, wherein:
a) the control device actuates the deflection device in such a way that at least two target elements are simultaneously irradiated by the particle beam.

12. Device according to claim 1, wherein:
a) the at least one support element includes, at least partially, a support material having a coefficient of thermal conductivity ≧10 W/(cm×K).

13. Device according to claim 1, wherein:
a) the at least one support element includes a support material, and the support material is one of diamond and contains diamond.

14. Device according to claim 1, wherein:
a) the at least one support element includes a support material, and the support material is doped to increase the electrical conductivity.

15. Device according to claim 1, wherein:
a) the target includes a filter which is permeable to the X-ray radiation generated in the target elements, and the filter at least partially blocks X-ray radiation generated in the support element.

16. Device according to claim 12, wherein:
a) the support material has a coefficient of thermal conductivity ≧20 W/(cm×K).

17. X-ray tomosynthesis device, comprising:
a) a target;
b) a device configured for directing a particle beam of electrically charged particles onto the target which emits X-ray radiation for irradiating a sample to be examined when the electrically charged particles strike the target, in use;
c) a radiation-sensitive detector, the radiation-sensitive detector being configured for recording irradiation images of radiation received at different radiation angles, the irradiation images recorded by the detector capable of being evaluated by use of computerized tomosynthesis image processing algorithms;
d) the target including at least one support element on which a plurality of mutually spaced target elements are provided, and each mutually spaced target element only partially covering the at least one support element; and
e) a deflection device provided, the deflection device being configured for causing the particle beam to be deflected in order to strike the plurality of mutually spaced target elements, in use.

18. Device according to claim 17, wherein:
a) a particle source is provided, the particle source being configured for generating the particle beam relative to the detector for detecting the X-ray radiation after examination of the sample, and the particle source is stationarily mounted at least during the irradiation of the sample.
